(19) United States
(12) Patent Application Publication
(10) Pub. No.: US 2022/0396619 A1
(43) Pub. Date: Dec. 15, 2022

(54) CD200 RECEPTOR ANTAGONIST BINDING MOLECULES

(71) Applicant: Eli Lilly and Company, Indianapolis, IN (US)

(72) Inventors: Katherine Marie BELL-MCGUINN, Zionsville, IN (US); Stephen John DEMAREST, San Diego, CA (US); Kyla Elizabeth DRISCOLL, Rutherford, NJ (US); Rikke Baek HOLMGAARD, West New York, NJ (US); Anna Marie RUSSELL, San Diego, CA (US); Jaafar Nassar SLEIMAN HAIDAR, Medfield, MA (US); Derrick Ryan WITCHER, Fishers, IN (US)

(21) Appl. No.: 17/774,214

(22) PCT Filed: Nov. 5, 2020
(86) PCT No.: PCT/US2020/059092

§ 371 Date: May 4, 2022

Related U.S. Application Data
(60) Provisional application No. 62/934,092, filed on Nov. 12, 2019.

(51) Int. Cl. C07K 16/28; A61K 39/395; A61P 35/00

(52) U.S. Cl. CPC C07K 16/2803; A61K 39/3955; A61P 35/00; A61K 2039/507

(57) ABSTRACT

The present invention relates to antagonist polypeptide molecules that bind to human CD200 receptor, and are useful for treating solid tumors, alone and in combination with chemotherapy, ionizing radiation, an antitumor agent and/or an immuno-oncology agent.

The present invention is in the field of medicine. More particularly, the present invention relates to antagonist polypeptide molecules that bind to human CD200 receptor (CD200R), compositions comprising such antagonist polypeptide molecules, and methods of using such antagonist polypeptide molecules for the treatment of cancer.

Immune checkpoint pathways suppress both the autoimmune response and the anti-cancer immune response (Isakov N, J. Autoimmune Disorders 2016; 2(2): 17). In autoimmune disease therapy, promoting, i.e., agonizing, the effect of an immune-suppressive pathway, such that the immune response is further suppressed, can be desirable. Conversely, in cancer therapy, inhibiting i.e., antagonizing, the effect of an immune-suppressive pathway, such that the immune response is derepressed, or stimulated, can be desirable.

The CD200 pathway is an immune-suppressive pathway, i.e., it restrains/suppresses the immune response (Rygiel T P and L Meyaard, Curr. Opin. Immunol 2012; 24: 233-238; Sun H, Immunology 2016; 178: 105-113), and CD200R is referred to as an inhibitory receptor (Hatherley D, et al., Structure 2013; 21: 820-832). Thus, a therapeutic molecule that promotes the immune-suppressive effect of the CD200 pathway is a CD200 pathway agonist (Gorczynski R M, ISRN Immunology 2012; Article ID 682168. doi.10.5402/2012/682168). Conversely, a therapeutic molecule that inhibits the immune-suppressive effect of the CD200 pathway is a CD200 pathway antagonist.

CD200 plays a pro-tumor role via direct inhibition of tumor reactive T cells and myeloid (e.g., mast) cells (Liu J-Q, et al., J. Immunol. 2016; 197: 1489-1497). Boosting the anti-cancer immune response can be an effective means of cancer therapy, and blocking CD200 ligand-CD200 receptor interaction is a potential therapeutic option to strengthen the immune system anti-cancer response (Rygiel T P and L Meyaard, Curr. Opin. Immunol 2012; 24: 233-238; Sun H, Immunology 2016; 178: 105-113).

Human CD200R is expressed as two “long isoform” alleles (AAQ89269.1 (ncbi.nlm.nih.gov/protein/AAQ89269.1); and (NP 620161.1 (ncbi.nlm.nih.gov/protein/NP 620161.1)), and two “short isoform” alleles (Q8TD46.2 (ncbi.nlm.nih.gov/protein/Q8TD46.2; and NP 740750 (ncbi.nlm.nih.gov/protein/NP_740750).

Some species, e.g., mouse and cynomolgus monkey, express the CD200RLa polypeptide, which shares sequence identity with CD200R, but CD200RLa is an activating receptor (Hatherley D, et al., Structure 2013; 21: 820-832), i.e., CD200RLa has the opposite effect on the immune response than does CD200R, and CD200RLa stimulation can result in mast cell activation (Zhang S and JH Phillips, J. Leukocyte Biol. 2005; 79: 363-368), which is undesirable.

Agonist anti-CD200R antibodies have been reported, e.g., Dx182 (U.S. Pat. No. 8,212,008). Samalizumab is an antagonist anti-CD200 ligand antibody (US 2005/0129690; U.S. Pat. No. 7,408,041; Mahadevan D, et al., Blood 2010; 116: 2465). A rabbit anti-mouse CD200R1 Fab has been reported (Gorczynski R M, et al., PLOS One 2014; 9(11): e113597).

However, no therapeutic CD200R-binding antagonist polypeptide molecules have been reported to be in development for human therapy. Thus, there remains a need for human CD200R-binding antagonist polypeptide molecules that bind to the long and short isoforms of human CD200R; block human CD200 ligand-human CD200R interaction; antagonize (derepress) the immune-suppressive effect of the CD200 pathway; bind to cynomolgus monkey CD200R; bind cynomolgus monkey CD200RLa, but do not elicit a mast cell degranulation response in vitro; do not elicit an unacceptable amount of mast cell activation in a primate model; or are useful in treating cancer.

Accordingly, the present invention provides polypeptide molecules that comprise each of the amino acid sequences of SEQ ID NOS: 1-6 (see Table 1) and that bind to the human CD200R long and short isoforms (SEQ ID NOS: 15 and 16, respectively) or to a human CD200R extracellular domain, (e.g., SEQ ID NO: 17); block human CD200 ligand-human CD200R interaction; antagonize the immune-suppressive effect of the CD200 pathway; bind cynomolgus monkey CD200R; bind cynomolgus monkey CD200RLa, but do not elicit a significant mast cell degranulation response in vitro; do not elicit an unacceptable amount of mast cell activation in a primate model; or demonstrate an anti-cancer effect in an in vivo model or in humans.

The amino acid sequences of SEQ ID NOS: 1-6 are fully human sequences. In one preferred embodiment, the polypeptide molecule amino acid residue sequence is fully human sequence.

In another preferred embodiment, the polypeptide molecule of the invention is human CD200R antagonist and also exhibits one or more of binding to the human CD200R long and short forms (SEQ ID NOS: 15 and 16, respectively); binding cynomolgus monkey CD200RLa, but without eliciting a significant mast cell degranulation response in an in vitro assay; elicits IL-2 release from Jurkat cells in an in vitro assay; and does not elicit an unacceptable amount of mast cell activation in a primate model.

The present invention also provides a polypeptide molecule that binds to the human CDw200R long and short isoforms, wherein the polypeptide molecule comprises each of the amino acid sequences of SEQ ID NOS: 1-6.

In another preferred embodiment, the polypeptide molecule of the invention is an scFv molecule. In another preferred embodiment, the polypeptide molecule of the invention is a Fab.

In another preferred embodiment, the polypeptide molecule of the invention is an antibody comprising: a heavy chain comprising an HCDR1 having the amino acid sequence of SEQ ID NO: 1, an HCDR2 having the amino acid sequence of SEQ ID NO: 2, and an HCDR3 having the amino acid sequence of SEQ ID NO: 3; and a light chain comprising an LCDR1 having the amino acid sequence of SEQ ID NO: 4, an LCDR2 having the amino acid sequence of SEQ ID NO: 5, and an LCDR3 having the amino acid sequence of SEQ ID NO: 6

In another preferred embodiment, the antibody is a mono-specific antibody. In another preferred embodiment, the antibody is a polyspecific antibody.

In another preferred embodiment, the polypeptide molecule of the invention is an antibody comprising a heavy chain variable region having the amino acid sequence of SEQ ID NO: 7 and a light chain variable region having the amino acid sequence of SEQ ID NO: 8. In another preferred embodiment, the polypeptide molecule is an antibody comprising a heavy chain having the amino acid sequence of SEQ ID NO: 11 and a light chain having the amino acid sequence of SEQ ID NO: 12.

The present invention also provides a polynucleotide molecule that encodes the polypeptide molecule of the invention. In one preferred embodiment, the polynucleotide (e.g., DNA) molecule comprises a polynucleotide encoding one or both of the polypeptide molecule amino acid sequences of SEQ ID NO: 11 and SEQ ID NO: 12. In another preferred embodiment, the polynucleotide molecule comprises polynucleotide sequence comprising one or both of SEQ ID NOS: 13 and 14.

The present invention also provides a mammalian cell capable of expressing the polypeptide molecule of the invention. The present invention also provides a mammalian cell comprising a polynucleotide (e.g., DNA) molecule of the invention. In one preferred embodiment, the polynucleotide molecule comprises a polynucleotide encoding one or both of the polypeptide molecule amino acid sequences of SEQ ID NO: 11 and SEQ ID NO: 12. In another preferred embodiment, the polynucleotide molecule comprises polynucleotide sequence comprising one or both of the polynucleotide sequences of SEQ ID NOS: 13 and 14.

The present invention also provides a process for producing a polypeptide molecule, comprising cultivating a mammalian cell of the invention, and recovering the polypeptide molecule. The present invention also provides the polypeptide molecule produced by the method.

The present invention also provides a pharmaceutical composition comprising a polypeptide molecule of the invention, and an acceptable carrier, diluent, or excipient.

The present invention also provides a method of treating a solid tumor, liquid tumor or neuroendocrine tumor cancer comprising administering to a human patient in need thereof an effective amount of a polypeptide molecule of the invention.

In one preferred embodiment, the solid tumor cancer is breast cancer, bladder cancer, cervical cancer, colorectal cancer, endometrial cancer, gastric cancer, head and neck cancer, hepatocellular carcinoma, liver cancer, lung cancer, melanoma, pancreatic cancer, prostate cancer, ovarian cancer, renal cancer, testicular cancer, or thyroid cancer. In another preferred embodiment, the solid tumor cancer is lung, breast or pancreatic cancer. In another preferred embodiment, the solid tumor cancer is lung cancer. In another preferred embodiment, the lung cancer is non-small cell lung cancer or small cell lung cancer. In another preferred embodiment, the solid tumor cancer is breast cancer. In another preferred embodiment, the breast cancer is triple-negative breast cancer, hormone receptor-positive/human epidermal growth factor-negative breast cancer. In another preferred embodiment, the solid tumor cancer pancreatic cancer.

In another preferred embodiment, the liquid tumor cancer is B-cell lymphoma, T-cell lymphoma, leukemia, Hodgkin lymphoma, myeloma, myelodysplasic syndrome, or plasmacytoma. In another preferred embodiment, the T-cell lymphoma is natural killer cell lymphoma. In another preferred embodiment, the leukemia is chronic lymphocytic leukemia, hairy cell leukemia, acute leukemia, lymphoblastic leukemia or myeloid leukemia. In another preferred embodiment, the leukemia is chronic lymphocytic leukemia. In another preferred embodiment, the lymphoblastic leukemia is acute lymphoblastic leukemia. In another preferred embodiment, the myeloid leukemia is acute myeloid leukemia or chronic myeloid leukemia. In another preferred embodiment, the myeloma is multiple myeloma.

In another preferred embodiment, the neuroendocrine tumor cancer is large cell neuroendocrine cancer or pancreatic neuroendocrine cancer.

In another preferred embodiment, the polypeptide molecule is administered in simultaneous, separate, or sequential combination with ionizing radiation. In another preferred embodiment, the polypeptide molecule is administered in simultaneous, separate, or sequential combination with one or more other anti-tumor agents. In another preferred embodiment, the polypeptide molecule is administered in simultaneous, separate, or sequential combination with ionizing radiation, and in simultaneous, separate, or sequential combination with one or more other anti-tumor agents.

The present invention also provides a polypeptide molecule of the invention, for use in therapy. In one preferred embodiment, the present invention provides a polypeptide of the invention for use in treating a solid tumor cancer, liquid tumor cancer or neuroendocrine tumor cancer.

In one embodiment, the solid tumor cancer is breast cancer, bladder cancer, cervical cancer, colorectal cancer, endometrial cancer, gastric cancer, head and neck cancer, hepatocellular carcinoma, liver cancer, lung cancer, melanoma, pancreatic cancer, prostate cancer, ovarian cancer, renal cancer, testicular cancer, or thyroid cancer. In another embodiment, the solid tumor cancer is lung, breast or pancreatic cancer. In another embodiment, the solid tumor cancer is lung cancer. In another embodiment, the lung cancer is non-small cell lung cancer or small cell lung cancer. In another embodiment, the solid tumor cancer is breast cancer. In another embodiment, the breast cancer is triple-negative breast cancer, hormone receptor-positive/human epidermal growth factor-negative breast cancer. In another embodiment, the solid tumor cancer pancreatic cancer.

In another embodiment, the liquid tumor cancer is B-cell lymphoma, T-cell lymphoma, leukemia, Hodgkin lymphoma, myeloma, myelodysplasic syndrome, or plasmacytoma. In another embodiment, the T-cell lymphoma is natural killer cell lymphoma. In another embodiment, the leukemia is chronic lymphocytic leukemia, hairy cell leukemia, acute leukemia, lymphoblastic leukemia or myeloid leukemia. In another embodiment, the leukemia is chronic lymphocytic leukemia. In another embodiment, the lymphoblastic leukemia is acute lymphoblastic leukemia. In another embodiment, the myeloid leukemia is acute myeloid leukemia or chronic myeloid leukemia. In another embodiment, the myeloma is multiple myeloma.

In another embodiment, the neuroendocrine tumor cancer is large cell neuroendocrine cancer or pancreatic neuroendocrine cancer.

In another embodiment, the polypeptide molecule is administered in simultaneous, separate, or sequential combination with ionizing radiation. In another embodiment, the polypeptide molecule is administered in simultaneous, separate, or sequential combination with one or more other anti-tumor agents. In another embodiment, the polypeptide molecule is administered in simultaneous, separate, or sequential combination with ionizing radiation, and in simultaneous, separate, or sequential combination with one or more other anti-tumor agents.

The present invention also provides for the use of a polypeptide molecule of the invention in the manufacture of a medicament for treating a solid tumor cancer, liquid tumor cancer or neuroendocrine tumor cancer.

In one embodiment, the solid tumor cancer is breast cancer, bladder cancer, cervical cancer, colorectal cancer, endometrial cancer, gastric cancer, head and neck cancer, hepatocellular carcinoma, liver cancer, lung cancer, melanoma, pancreatic cancer, prostate cancer, ovarian cancer, renal cancer, testicular cancer, or thyroid cancer. In another embodiment, the solid tumor cancer is lung, breast or pancreatic cancer. In another embodiment, the solid tumor cancer is lung cancer. In another embodiment, the lung cancer is non-small cell lung cancer or small cell lung cancer. In another embodiment, the solid tumor cancer is breast cancer. In another embodiment, the breast cancer is triple-negative breast cancer, hormone receptor-positive/human epidermal growth factor-negative breast cancer. In another embodiment, the solid tumor cancer pancreatic cancer.

In another embodiment, the liquid tumor cancer is B-cell lymphoma, T-cell lymphoma, leukemia, Hodgkin lymphoma, myeloma, myelodysplasic syndrome, or plasmacytoma. In another embodiment, the T-cell lymphoma is natural killer cell lymphoma. In another embodiment, the leukemia is chronic lymphocytic leukemia, hairy cell leukemia, acute leukemia, lymphoblastic leukemia or myeloid leukemia. In another embodiment, the leukemia is chronic lymphocytic leukemia. In another embodiment, the lymphoblastic leukemia is acute lymphoblastic leukemia. In another embodiment, the myeloid leukemia is acute myeloid leukemia or chronic myeloid leukemia. In another embodiment, the myeloma is multiple myeloma.

In another preferred embodiment, the neuroendocrine tumor cancer is large cell neuroendocrine cancer or pancreatic neuroendocrine cancer.

In an embodiment, the polypeptide molecule is administered in simultaneous, separate, or sequential combination with ionizing radiation. In an embodiment, the polypeptide molecule is administered in simultaneous, separate, or sequential combination with one or more other anti-tumor agents. In an embodiment, the polypeptide molecule is administered in simultaneous, separate, or sequential combination with ionizing radiation, and in simultaneous, separate, or sequential combination with one or more other anti-tumor agents.

The present invention also provides an antibody that binds to the human CD200R long and short isoforms (SEQ ID NOS: 15 and 16, respectively), or to a CD200R extracellular domain, (e.g., SEQ ID NO: 17), comprising:
a) an HCDR1 having the amino acid sequence of SEQ ID NO:1, an HCDR2 having the amino acid sequence of SEQ ID NO:2, and an HCDR3 having the amino acid sequence of SEQ ID NO:3; and
b) an LCDR1 having the amino acid sequence of SEQ ID NO:4, an LCDR2 having the amino acid sequence of SEQ ID NO:5, and an LCDR3 having the amino acid sequence of SEQ ID NO:6.

The present invention also provides an antibody comprising:
a) a heavy chain variable region having the amino acid sequence of SEQ ID NO: 7; and
b) a light chain variable region having the amino acid sequence of SEQ ID NO: 8.

The present invention also provides an antibody comprising: (a) a heavy chain having the amino acid sequence of SEQ ID NO: 11; and (b) a light chain having the amino acid sequence of SEQ ID NO: 12.

In one preferred embodiment, the heavy chain of the antibody forms at least one disulfide bond with the light chain of the antibody, and the two heavy chains of the antibody form at least one disulfide bond.

In another preferred embodiment, the antibody is a human IgG1 engineered to reduce the binding of the antibody to an Fc gamma receptor.

The present invention also provides a process for producing an antibody comprising cultivating a mammalian cell comprising a polynucleotide encoding the antibody and capable of expressing the antibody, and recovering the antibody, the antibody comprising:
a) an HCDR1 having the amino acid sequence of SEQ ID NO: 1, an HCDR2 having the amino acid sequence of SEQ ID NO: 2, and an HCDR3 having the amino acid sequence of SEQ ID NO: 3; and
b) an LCDR1 having the amino acid sequence of SEQ ID NO: 4, an LCDR2 having the amino acid sequence of SEQ ID NO: 5, and an LCDR3 having the amino acid sequence of SEQ ID NO: 6.

In one preferred embodiment, the heavy chain of the antibody forms at least one disulfide bond with the light chain of the antibody, and the two heavy chains of the antibody form at least one disulfide bond.

The present invention also provides a process for producing an antibody comprising cultivating a mammalian cell comprising a polynucleotide encoding the antibody and capable of expressing the antibody and recovering the antibody, the antibody comprising:
a) a heavy chain variable region having the amino acid sequence of SEQ ID NO: 7; and
b) a light chain variable region having the amino acid sequence of SEQ ID NO: 8.

The present invention also provides a process for producing an antibody comprising cultivating a mammalian cell capable of expressing the antibody and recovering the antibody, the antibody comprising:
a) a heavy chain having the amino acid sequence of SEQ ID NO: 11; and
b) a light chain having the amino acid sequence of SEQ ID NO: 12.

The present invention also provides a process for producing an antibody comprising cultivating a mammalian cell comprising a polynucleotide encoding the antibody and capable of expressing the antibody and recovering the antibody; wherein the antibody is a human IgG1 engineered to reduce the binding of the antibody to an Fc gamma receptor.

The present invention also provides an antibody produced by a process comprising cultivating a mammalian cell capable of expressing the antibody and recovering the antibody, the antibody comprising:
a) an HCDR1 having the amino acid sequence of SEQ ID NO: 1, an HCDR2 having the amino acid sequence of SEQ ID NO: 2, and an HCDR3 having the amino acid sequence of SEQ ID NO: 3; and
b) an LCDR1 having the amino acid sequence of SEQ ID NO: 4, an LCDR2 having the amino acid sequence of SEQ ID NO: 5, and an LCDR3 having the amino acid sequence of SEQ ID NO: 6.

In one embodiment, the heavy chain of the antibody produced forms at least one disulfide bond with the light chain of the antibody, and the two heavy chains of the antibody form at least one disulfide bond.

The present invention also provides an antibody produced by a process comprising cultivating a mammalian cell capable of expressing the antibody and recovering the antibody, comprising:
a) a heavy chain variable region having the amino acid sequence of SEQ ID NO: 7;
b) a light chain variable region having the amino acid sequence of SEQ ID NO: 8.

The present invention also provides an antibody produced by a process comprising cultivating a mammalian cell capable of expressing the antibody and recovering the antibody, comprising:
a) a heavy chain having the amino acid sequence of SEQ ID NO: 11;
b) a light chain having the amino acid sequence of SEQ ID NO: 12.

The present invention also provides a pharmaceutical composition comprising an antibody, wherein the antibody binds to the human CD200R long and short isoforms (SEQ ID NOS: 15 and 16, respectively), or to a human CD200R extracellular domain, (e.g., SEQ ID NO: 17), comprising:
a) an HCDR1 having the amino acid sequence of SEQ ID NO: 1, an HCDR2 having the amino acid sequence of SEQ ID NO: 2, and an HCDR3 having the amino acid sequence of SEQ ID NO: 3; and
b) an LCDR1 having the amino acid sequence of SEQ ID NO: 4, an LCDR2 having the amino acid sequence of SEQ ID NO: 5, and an LCDR3 having the amino acid sequence of SEQ ID NO: 6, and an acceptable carrier, diluent, or excipient.

In one preferred embodiment, the heavy chain of the antibody forms at least one disulfide bond with the light chain of the antibody, and the two heavy chains of the antibody form at least one disulfide bond.

The present invention also provides a pharmaceutical composition comprising an antibody comprising:
a) a heavy chain variable region having the amino acid sequence of SEQ ID NO: 7;
b) a light chain variable region having the amino acid sequence of SEQ ID NO: 8, and an acceptable carrier, diluent, or excipient.

The present invention also provides a pharmaceutical composition comprising an antibody comprising:
a) a heavy chain having the amino acid sequence of SEQ ID NO: 11;
b) a light chain having the amino acid sequence of SEQ ID NO: 12, and an acceptable carrier, diluent, or excipient.

In one preferred embodiment, antibody is a human IgG1 engineered to reduce the binding of the antibody to an Fc gamma receptor.

The present invention also provides a method of treating a solid tumor cancer, liquid tumor cancer or neuroendocrine cancer, comprising administering to a human patient in need thereof, an effective amount of an antibody, wherein the antibody binds to the human CD200R long and short isoforms (SEQ ID NOS: 15 and 16, respectively), or a human CD200R extracellular domain, (e.g., SEQ ID NO: 17), comprising:
a) an HCDR1 having the amino acid sequence of SEQ ID NO: 1, an HCDR2 having the amino acid sequence of SEQ ID NO: 2, and an HCDR3 having the amino acid sequence of SEQ ID NO: 3; and
b) an LCDR1 having the amino acid sequence of SEQ ID NO: 4, an LCDR2 having the amino acid sequence of SEQ ID NO: 5, and an LCDR3 having the amino acid sequence of SEQ ID NO: 6.

In one preferred embodiment, the heavy chain of the antibody forms at least one disulfide bond with the light chain of the antibody, and the two heavy chains of the antibody form at least one disulfide bond.

The present invention also provides a method of treating a solid tumor cancer, liquid tumor cancer or neuroendocrine cancer comprising administering to a human patient in need thereof, an effective amount of an antibody, the antibody comprising:
a) a heavy chain variable region having the amino acid sequence of SEQ ID NO: 7; and
b) a light chain variable region having the amino acid sequence of SEQ ID NO: 8.

The present invention also provides a method of treating cancer comprising administering to a human patient in need thereof, an effective amount of an antibody, the antibody comprising:
a) a heavy chain having the amino acid sequence of SEQ ID NO: 11; and
b) a light chain having the amino acid sequence of SEQ ID NO: 12.

In one preferred embodiment, the antibody is a human IgG1 engineered to reduce the binding of the antibody to an Fc gamma receptor.

In another preferred embodiment, the polypeptide molecule is administered in simultaneous, separate, or sequential combination with ionizing radiation. In another preferred embodiment, the polypeptide molecule is administered in simultaneous, separate, or sequential combination with one or more other anti-tumor agents. In another preferred embodiment, the polypeptide molecule is administered in simultaneous, separate, or sequential combination with ionizing radiation, and in simultaneous, separate, or sequential combination with one or more other anti-tumor agents.

In one embodiment, the present invention also provides a method of treating cancer, comprising administering an effective amount of a polypeptide molecule disclosed herein in simultaneous, separate, or sequential combination with one or more other anti-tumor agents. Non-limiting examples of anti-tumor agents include ramucirumab, necitumumab, gemcitabine, pemetrexed, galunisertib, abemaciclib, cisplatin, carboplatin, dacarbazine, liposomal doxorubicin, docetaxel, cyclophosphamide and doxorubicin, navelbine, eribulin, paclitaxel, paclitaxel protein-bound particles for injectable suspension, ixabepilone, capecitabine, FOLFOX (leucovorin, fluorouracil, and oxaliplatin), FOLFIRI (leucovorin, fluorouracil, and irinotecan), cetuximab, an EGFR inhibitor, a Raf inhibitor, a B-Raf inhibitor, a CDK4/6 inhibitor, a CDK7 inhibitor, an idoleamine 2,3-dioxygenase inhibitor, a TGFI3 inhibitor, a TGFI3 receptor inhibitor, IL-10, and pegylated IL-10 (e.g., pegilodecakin).

In a another embodiment, the present invention provides a method of treating cancer, comprising administering an effective amount of a compound of a polypeptide molecule of the invention in simultaneous, separate, or sequential combination with one or more immuno-oncology agents. Non-limiting examples of immuno-oncology agents include nivolumab, ipilimumab, pidilizumab, pembrolizumab, tremelimumab, urelumab, lirilumab, atezolizumab, durvalumab, an anti-Tim3 antibody, an anti-PD-1 antibody, an anti-PD-L1 antibody. In another preferred embodiment, the immuno-oncology agent is an anti-PD-1 antibody or an anti-PD-1 antibody. In another preferred embodiment, the anti-PD-1 antibody is pembrolizumab. In another preferred embodiment, the anti-PD-L1 antibody is LY3300054 (the heavy and light chain sequences of which are forth in WO 2017/034916 and US 2017/0058033 as SEQ ID NOS: 10 and 11, respectively).

The present invention also provides an antibody for use in treating cancer, wherein the antibody binds to the human CD200R long and short isoforms (SEQ ID NOS: 15 and 16, respectively), or to a human CD200R extracellular domain, (e.g., SEQ ID NO: 17), the antibody comprising:
a) an HCDR1 having the amino acid sequence of SEQ ID NO: 1, an HCDR2 having the amino acid sequence of SEQ ID NO: 2, and an HCDR3 having the amino acid sequence of SEQ ID NO: 3; and
b) an LCDR1 having the amino acid sequence of SEQ ID NO: 4, an LCDR2 having the amino acid sequence of SEQ ID NO: 5, and an LCDR3 having the amino acid sequence of SEQ ID NO: 6.

In one preferred embodiment, the heavy chain of the antibody forms at least one disulfide bond with the light chain of the antibody, and the two heavy chains of the antibody form at least one disulfide bond.

The present invention also provides an antibody for use in treating a solid tumor cancer, a liquid tumor cancer or a neuroendocrine cancer, the antibody comprising:
a) a heavy chain variable region having the amino acid sequence of SEQ ID NO: 7; and
b) a light chain variable region having the amino acid sequence of SEQ ID NO: 8.

The present invention also provides an antibody for use in treating cancer, comprising:
a) a heavy chain having the amino acid sequence of SEQ ID NO: 11; and
b) a light chain having the amino acid sequence of SEQ ID NO: 12.

In one preferred embodiment, the antibody is a human IgG1 engineered to reduce the binding of the antibody to an Fc gamma receptor.

In one embodiment, the solid tumor cancer is breast cancer, bladder cancer, cervical cancer, colorectal cancer, endometrial cancer, gastric cancer, head and neck cancer, hepatocellular carcinoma, liver cancer, lung cancer, melanoma, pancreatic cancer, prostate cancer, ovarian cancer, renal cancer, testicular cancer, or thyroid cancer. In another embodiment, the solid tumor cancer is lung, breast or pancreatic cancer. In another embodiment, the solid tumor cancer is lung cancer. In another embodiment, the lung cancer is non-small cell lung cancer or small cell lung cancer. In another embodiment, the solid tumor cancer is breast cancer. In another embodiment, the breast cancer is triple-negative breast cancer, hormone receptor-positive/human epidermal growth factor-negative breast cancer. In another embodiment, the solid tumor cancer pancreatic cancer.

In another embodiment, the liquid tumor cancer is B-cell lymphoma, T-cell lymphoma, leukemia, Hodgkin lymphoma, myeloma, myelodysplasic syndrome, or plasmacytoma. In an embodiment, the T-cell lymphoma is natural killer cell lymphoma. In an embodiment, the leukemia is chronic lymphocytic leukemia, hairy cell leukemia, acute leukemia, lymphoblastic leukemia or myeloid leukemia. In an embodiment, the leukemia is chronic lymphocytic leukemia. In an embodiment, the lymphoblastic leukemia is acute lymphoblastic leukemia. In an embodiment, the myeloid leukemia is acute myeloid leukemia or chronic myeloid leukemia. In an embodiment, the myeloma is multiple myeloma.

In another preferred embodiment, the neuroendocrine tumor cancer is large cell neuroendocrine cancer or pancreatic neuroendocrine cancer.

In one preferred embodiment, the antibody of the invention is administered in simultaneous, separate, or sequential combination with ionizing radiation. In another preferred embodiment, the antibody of the invention is administered in simultaneous, separate, or sequential combination with one or more other anti-tumor agents. In another preferred embodiment, the antibody of the invention is administered in simultaneous, separate, or sequential combination with ionizing radiation and one or more other anti-tumor agents.

The present invention also provides a pharmaceutical composition comprising an antibody for use in treating cancer, wherein the antibody binds to the human CD200R long and short isoforms (SEQ ID NOS: 15 and 16, respectively), or a human CD200R extracellular domain, (e.g., SEQ ID NO: 17), wherein the antibody comprises:
a) an HCDR1 having the amino acid sequence of SEQ ID NO: 1, an HCDR2 having the amino acid sequence of SEQ ID NO: 2, and an HCDR3 having the amino acid sequence of SEQ ID NO: 3; and
b) an LCDR1 having the amino acid sequence of SEQ ID NO: 4, an LCDR2 having the amino acid sequence of SEQ ID NO: 5, and an LCDR3 having the amino acid sequence of SEQ ID NO: 6, and an acceptable carrier, diluent, or excipient.

In one preferred embodiment, the heavy chain of the antibody forms at least one disulfide bond with the light chain of the antibody, and the two heavy chains of the antibody form at least one disulfide bond.

The present invention also provides a pharmaceutical composition comprising an antibody for use in treating a solid tumor cancer, a liquid tumor cancer or a neuroendocrine cancer, wherein the antibody comprises:
a) a heavy chain variable region having the amino acid sequence of SEQ ID NO: 7; and
b) a light chain variable region having the amino acid sequence of SEQ ID NO: 8,
and an acceptable carrier, diluent, or excipient.

The present invention also provides a pharmaceutical composition comprising an antibody for use in treating a solid tumor cancer, a liquid tumor cancer or a neuroendocrine cancer, wherein the antibody comprises:
a) a heavy chain having the amino acid sequence of SEQ ID NO: 11; and
b) a light chain having the amino acid sequence of SEQ ID NO: 12,
and an acceptable carrier, diluent, or excipient.

In one preferred embodiment, the antibody is a human IgG1 engineered to reduce the binding of the antibody to an Fc gamma receptor.

In one embodiment, the solid tumor cancer is breast cancer, bladder cancer, cervical cancer, colorectal cancer, endometrial cancer, gastric cancer, head and neck cancer, hepatocellular carcinoma, liver cancer, lung cancer, melanoma, pancreatic cancer, prostate cancer, ovarian cancer, renal cancer, testicular cancer, or thyroid cancer. In another embodiment, the solid tumor cancer is lung, breast or pancreatic cancer. In another embodiment, the solid tumor cancer is lung cancer. In another embodiment, the lung cancer is non-small cell lung cancer or small cell lung cancer. In another embodiment, the solid tumor cancer is breast cancer. In another embodiment, the breast cancer is triple-negative breast cancer, hormone receptor-positive/human epidermal growth factor-negative breast cancer. In another embodiment, the solid tumor cancer pancreatic cancer.

In another embodiment, the liquid tumor cancer is B-cell lymphoma, T-cell lymphoma, leukemia, Hodgkin lymphoma, myeloma, myelodysplasic syndrome, or plasmacytoma. In an embodiment, the T-cell lymphoma is natural killer cell lymphoma. In an embodiment, the leukemia is chronic lymphocytic leukemia, hairy cell leukemia, acute leukemia, lymphoblastic leukemia or myeloid leukemia. In an embodiment, the leukemia is chronic lymphocytic leukemia. In an embodiment, the lymphoblastic leukemia is acute lymphoblastic leukemia. In an embodiment, the myeloid leukemia is acute myeloid leukemia or chronic myeloid leukemia. In an embodiment, the myeloma is multiple myeloma.

In another preferred embodiment, the neuroendocrine tumor cancer is large cell neuroendocrine cancer or pancreatic neuroendocrine cancer.

In one embodiment, the composition is administered in simultaneous, separate, or sequential combination with ionizing radiation. In another embodiment, the pharmaceutical composition is administered in simultaneous, separate, or sequential combination with one or more other anti-tumor agents. In another embodiment, the pharmaceutical composition is administered in simultaneous, separate, or sequential combination with ionizing radiation and one or more other anti-tumor agents.

The present invention also provides the use of an antibody of the present invention in the manufacture of a medicament for treating a solid tumor cancer, a liquid tumor cancer, or a neuroendocrine tumor cancer, wherein the antibody binds to the human CD200R long and short isoforms (SEQ ID NOS: 15 and 16, respectively), or to a human CD200R extracellular domain, (e.g., SEQ ID NO: 17), comprising:
a) an HCDR1 having the amino acid sequence of SEQ ID NO: 1, an HCDR2 having the amino acid sequence of SEQ ID NO: 2, and an HCDR3 having the amino acid sequence of SEQ ID NO: 3; and
b) an LCDR1 having the amino acid sequence of SEQ ID NO: 4, an LCDR2 having the amino acid sequence of SEQ ID NO: 5, and an LCDR3 having the amino acid sequence of SEQ ID NO: 6.

In one embodiment, the heavy chain of the antibody forms at least one disulfide bond with the light chain of the antibody, and the two heavy chains of the antibody form at least one disulfide bond.

The present invention also provides the use of an antibody of the present invention in the manufacture of a medicament for treating cancer, comprising:
a) a heavy chain variable region having the amino acid sequence of SEQ ID NO: 7; and
b) a light chain variable region having the amino acid sequence of SEQ ID NO: 8.

The present invention also provides the use of an antibody of the present invention in the manufacture of a medicament for treating cancer, comprising:
a) a heavy chain having the amino acid sequence of SEQ ID NO: 11; and
b) a light chain having the amino acid sequence of SEQ ID NO: 12.

In one preferred embodiment, the antibody is a human IgG1 engineered to reduce the binding of the antibody to an Fc gamma receptor.

In one embodiment, the solid tumor cancer is breast cancer, bladder cancer, cervical cancer, colorectal cancer, endometrial cancer, gastric cancer, head and neck cancer, hepatocellular carcinoma, liver cancer, lung cancer, melanoma, pancreatic cancer, prostate cancer, ovarian cancer, renal cancer, testicular cancer, or thyroid cancer. In another embodiment, the solid tumor cancer is lung, breast or pancreatic cancer. In another embodiment, the solid tumor cancer is lung cancer. In another embodiment, the lung cancer is non-small cell lung cancer or small cell lung cancer. In another embodiment, the solid tumor cancer is breast cancer. In another embodiment, the breast cancer is triple-negative breast cancer, hormone receptor-positive/human epidermal growth factor-negative breast cancer. In another embodiment, the solid tumor cancer pancreatic cancer.

In another embodiment, the liquid tumor cancer is B-cell lymphoma, T-cell lymphoma, leukemia, Hodgkin lymphoma, myeloma, myelodysplasic syndrome, or plasmacytoma. In an embodiment, the T-cell lymphoma is natural killer cell lymphoma. In an embodiment, the leukemia is chronic lymphocytic leukemia, hairy cell leukemia, acute leukemia, lymphoblastic leukemia or myeloid leukemia. In an embodiment, the leukemia is chronic lymphocytic leukemia. In an embodiment, the lymphoblastic leukemia is acute lymphoblastic leukemia. In an embodiment, the myeloid leukemia is acute myeloid leukemia or chronic myeloid leukemia. In an embodiment, the myeloma is multiple myeloma.

In another preferred embodiment, the neuroendocrine tumor cancer is large cell neuroendocrine cancer or pancreatic neuroendocrine cancer.

In one embodiment, the antibody is administered in simultaneous, separate, or sequential combination with ionizing radiation. In another preferred embodiment, the antibody is administered in simultaneous, separate, or sequential combination with one or more other anti-tumor agents. In another preferred embodiment, the antibody is administered in simultaneous, separate, or sequential combination with ionizing radiation and one or more other anti-tumor agents.

In one preferred embodiment, the polypeptide molecule of the invention is sterile. In another embodiment, the polypeptide molecule of the invention is substantially pure. In another embodiment, the polypeptide molecule of the invention is substantially pure and sterile.

The polypeptide molecules of the present invention, or pharmaceutical compositions comprising the same, may be administered by a parenteral route. In one preferred embodiment, administration is intravenous. In another preferred embodiment, administration is subcutaneous.

The polypeptide molecules of the present invention may be administered to a human patient alone with pharmaceutically acceptable carriers, diluents, or excipients in single or multiple doses. A pharmaceutical composition of the present invention may be prepared by methods known in the art (e.g., Remington: The Science and Practice of Pharmacy, 22^{nd} ed. (2012), A. Loyd et al., Pharmaceutical Press).

Dosage regimens for administering a polypeptide molecule of the invention may be adjusted to provide the optimum desired response (e.g., a therapeutic effect).

The term “polypeptide molecule” as used herein refers to a molecule that comprises a polymer of amino acid residues. In another preferred embodiment, the polypeptide molecule consists of a polymer of amino acid residues.

The term “antibody” as used herein refers to a monomeric or dimeric immunoglobulin molecule having a heavy chain and a light chain that recognizes and binds to a target, such as a protein, peptide or polypeptide. In one embodiment, the antibody specifically binds to the target. Each heavy chain is comprised of an N-terminal HCVR (heavy chain variable region) and an HCCR (heavy chain constant region). Each light chain is comprised of an N-terminal LCVR (light chain variable region) and a LCCR (light chain constant region). The constant region of the heavy chains contain CH1, CH2, and CH3 domains.

As used herein, “Antibody A” refers to an antibody having a heavy chain having the amino acid sequence of SEQ ID NO: 11 and light chain having the amino acid sequence of SEQ ID NO: 12

Human IgG1 is known to bind to the proteins of the Fc-gamma receptor (FcγR) family as well as C1q. IgG1 binding to an FcγR or C1q induces antibody-dependent cellular cytotoxicity (ADCC) and complement-dependent cytotoxicity (CDC), respectively. In one preferred embodiment, the antibodies described herein are a human IgG1 engineered to reduce the binding of the antibody to an FcγR as well as C1q. In another preferred embodiment, amino acid substitutions of positions L234A, L235A and P329A in EU numbering are introduced into the CH2 region to reduce the binding of the antibody to an FcγR as well as C1q. Optionally, amino acid substitution of position N297Q in EU numbering is introduced to further reduce the ADCC and CDC activities of the antibody.

The term “modified human IgG1” as used herein means a human IgG1 engineered to reduce the binding of the human IgG1 to at least one human Fc gamma receptor. Typically this is performed by mutating residues that lead to a reduction in the binding of the antibody to the Fc gamma receptor(s), e.g., P329A, L234A and L235 A mutations.

The term “IgG4-PAA” refers to an IgG4 molecule in which the Fc portion has a serine to proline mutation at position 227 (S227P); a phenylalanine to alanine mutation at position 233 (F233A); and a leucine to alanine mutation at position 234 (L234A).

The term “binds” as used herein refers to the molecular interaction between two molecules, e.g., a polypeptide molecule of the invention and CD200R. The term “monospecific binding” refers to binding to one target, e.g., human. The term “bispecific binding” refers to binding to human CD200R and to another target. The term “polyspecific binding” refers to binding to human CD200R and to two other targets.

In one preferred embodiment, the polypeptide molecule of the present invention binds specifically to human CD200R, or to an extracellular domain thereof. In one preferred embodiment, “specifically binds” means that a polypeptide molecule of the invention interacts more frequently, more rapidly, with greater duration, with greater affinity, or with some combination of the above to human CD200R. In another preferred embodiment, “specifically binds” means that a polypeptide molecule of the invention binds to human CD200R with a K_{D} of about 0.1 mM or less. In another preferred embodiment, “specifically binds” means that a polypeptide molecule of the invention binds to human CD200R with a K_{D} of about 0.01 mM or less. In another preferred embodiment, “specifically binds” means that a polypeptide molecule of the invention binds to human CD200R with a K_{D} of about 0.001 mM or less. In another preferred embodiment, “specifically binds” means that a polypeptide molecule of the invention binds to human CD200R with a K_{D} of about 0.0001 mM or less.

Unless otherwise indicated herein, “CD200R” refers to human CD200R.

Synonyms for CD200R are CD200R1, OX2R, MOX2R and HCRTR2.

The term “substantially pure” refers to having been separated from other materials. In one preferred embodiment, “substantially pure” means 80, 85, 90, 95, 96, 97, 98 or 99% pure.

The term “treating” (or “treat” or “treatment”) refers to slowing, interrupting, arresting, alleviating, stopping, reducing, or reversing the progression or severity of an existing symptom, disorder, condition, or disease.

The term “effective amount” means the amount of a polypeptide molecule of the present invention or a pharmaceutical composition comprising a polypeptide molecule of the invention that elicits the biological or medical response or desired therapeutic effect on a tissue, system, animal, mammal or human that is being sought by the researcher, medical doctor, or other clinician. An effective amount of the polypeptide molecule may vary according to factors such as the disease state, age, sex, and weight of the individual, and the ability of the polypeptide molecule to elicit a desired response in the individual. An effective amount is also one in which any toxic or detrimental effect of the antibody is outweighed by the therapeutically beneficial effects.

An isolated polynucleotide molecule encoding a HCVR region may be converted to a full-length heavy chain gene by operably linking the HCVR-encoding polynucleotide to another polynucleotide molecule encoding heavy chain constant regions. The sequences of human, as well as other mammalian, heavy chain constant region genes are known in the art. polynucleotide fragments encompassing these regions may be obtained, e.g., by standard PCR amplification.

An isolated polynucleotide molecule encoding a LCVR region may be converted to a full-length light chain gene by operably linking the LCVR-encoding polynucleotide to another polynucleotide molecule encoding a light chain constant region. The sequences of human, as well as other mammalian, light chain constant region genes are known in the art. Polynucleotide fragments encompassing these regions may be obtained by standard PCR amplification.

As used herein, the term “CDR” refers to an antibody complementarity determining region, the term “HCDR” refers to an antibody heavy chain CDR, and the term “LCDR” refers to an antibody light chain CDR. For the purposes of the present invention, the framework and CDR sequences in each of the antibodies for which sequences are set forth herein are annotated using annotation rules in agreement with the method of the North CDR definitions are used (North et al., “A New Clustering of Antibody CDR Loop Conformations”, Journal of Molecular Biology, 406, 228-256 (2011).

The term “solid tumor” refers to a tumor in a tissue that is not blood, lymphatics or bone marrow. The term “liquid tumor” refers to a tumor in a tissue that originates in the blood, lymphatics or bone marrow. The term “neuroendocrine tumor” refers to a tumor that originates in a neuroendocrine tissue.

As used herein, “CD200R antagonist” refers to a polypeptide molecule that binds to CD200R and inhibits the immune-suppressive effect of the CD200/CD200R pathway. CD200R recruits the adaptor protein downstream of tyrosine kinase 2 (Dok2), which in turn recruits its target RAS p21 protein activator 1 (Ras-GAP), leading to inhibition of ERK, JNK, and p38 MAPK activation (Mukhopadhyay S, et al., Cell Host & Microbe 2010; 8: 236-247). Methods for assaying CD200R activity in vitro, e.g., using a Dok2 phosphorylation assay, are known to those of ordinary skill in the art (Mihrshahi R, et al., J. Immunology 2009; 183: 4879-4886; Mihrshahi R and M H Brown, J. Immunology 2010; 185: 7216-7222). Thus, a CD200R antagonist is a polypeptide molecule that reduces the level of Dok2 phosphorylation (pDok2), relative to the level of Dok2 phosphorylation observed in a control experiment performed in the absence of the CD200R antagonist.

In vivo murine models of solid tumor are well known to those of ordinary skill in the art, as shown herein, and as disclosed, e.g., in Sanmamed N W, et al., Ann. Oncol. 2016; 27: 1190-1198; Manning H C, et al., J. Nucl. Med 2016; 57 (Suppl. 1): 60S-68S; Teich B A. Cancer Ther. 2006; 5: 2435; Rongvaux A, et al., Ann. Rev. Immunol. 2013; 31: 635-74; Stylli S S, et al., J. Clin. Neurosci 2015; 619-26; Oh T, et al., J. Transl. Med. 2014; 12: 107-117; Newcomb, E W, et al., Radiation Res. 2010; 173: 426-432; Song Y, et al., Proc Natl. Acad. Sci. USA 2013; 110: 17933-8; and Rutter E M, et al., Scientific Reports 2017; 7: DOI: 10.1038/s41598-017-02462-0.

The polynucleotides of the present invention may be expressed in a host cell after the sequences are operably linked to an expression control sequence. The expression vectors are typically replicable in the host organisms either as episomes or as an integral part of the host chromosomal DNA. Commonly, expression vectors contain selection markers, e.g., tetracycline, neomycin, and dihydrofolate reductase, to permit detection of those cells transformed with the desired polynucleotide sequences.

An expression vector containing the polynucleotide sequences of interest (e.g., the polynucleotides encoding the polypeptides of a polypeptide molecule and expression control sequences) can be transferred into a host cell by known methods, which vary depending on the type of host cells.

A polypeptide molecule of the present invention may be produced in mammalian host cells, non-limiting examples of which include CHO, NSO, HEK293 or COS cells. The host cells may be cultured using techniques known in the art.

Various methods of protein purification may be employed to purify an antibody of the present invention and such methods are known in the art and described, for example, in Deutscher, Methods in Enzymology 182: 83-89 (1990) and Scopes, Protein Purification: Principles and Practice, 3rd Edition, Springer, N.Y. (1994). Sequences referred to herein are numbered according to the sequence identifier numbers listed in Table 1.

TABLE 1
Sequence identifier numbers

| | | |
|---|---|---|
| Anti-human | HCDR1 (AA) | 1 |
| CD200R | HCDR2 (AA) | 2 |
| (Antibody A) | HCDR3 (AA) | 3 |
| | LCDR1 (AA) | 4 |
| | LCDR2 (AA) | 5 |
| | LCDR3 (AA) | 6 |
| | HCVR (AA) | 7 |
| | LCVR (AA) | 8 |
| | HCCR (AA) | 9 |
| | LCCR (AA) | 10 |
| | Heavy chain (AA) | 11 |
| | Light chain (AA) | 12 |
| | Heavy Chain (DNA) | 13 |
| | Light Chain (DNA) | 14 |

| | |
|---|---|
| Human CD200R long isoform (AA) | 15 |
| Human CD200R short isoform (AA) | 16 |
| Human CD200R long isoform ECD-His (AA) | 17 |
| Cynomolgus monkey CD200R long isoform (AA) | 18 |
| Cynomolgus monkey CD200R long isoform ECD- | 19 |
| His (AA) | |
| Cynomolgus monkey CD200RLa (AA) | 20 |

AA: amino acid;
HCDR: heavy chain CDR;
LCDR: light chain CDR;
HCCR: heavy chain constant region;
LCCR: light chain constant region;
HCVR: heavy chain variable region;
LCVR: light chain variable region;
ECD: extracellular domain;
ECD-His: extracellular domain-Histidine;
HC: heavy chain;
LC: light chain

### Antibody Expression and Purification

The antibodies of the present invention may be expressed and purified essentially as follows. An appropriate host cell, such as HEK 293 or CHO, may be either transiently or stably transfected with an expression system for secreting antibodies using an optimal predetermined heavy chain:light chain vector ratio or a single vector system encoding both heavy chain and light chain. Antibody A of the present invention may be either transiently or stably transfected with an expression system for secreting antibodies using one or more DNA molecules encoding for a heavy chain having the amino acid sequence of SEQ ID NO: 11, and light chain having the amino acid sequence of SEQ ID NO: 12, e.g., SEQ ID NOS: 13 and 14, respectively.

The antibodies may be purified using one of many commonly-used techniques. For example, the medium may be conveniently applied to a Mab Select column (GE Healthcare Life Sciences), or KappaSelect column (GE Healthcare Life Sciences), that has been equilibrated with a compatible buffer, such as phosphate buffered saline (pH 7.4). The column may be washed to remove nonspecific binding components. The bound antibody may be eluted, for example, by pH gradient (such as 20 mM Tris buffer pH 7.0 to 10 mM sodium citrate buffer pH 3.0, or phosphate buffered saline pH 7.4 to 100 mM glycine buffer pH 3.0). Antibody fractions may be detected, such as by UV absorbance or SDS-PAGE, and then may be pooled. Further purification is optional, depending on the intended use. The purified antibody may be concentrated and/or sterile filtered using common techniques. Soluble aggregate and multimers may be effectively removed by common techniques, including size exclusion, hydrophobic interaction, ion exchange, multimodal, or hydroxyapatite chromatography. The purified antibody may be immediately frozen at −70° C. or may be lyophilized.

### Antibody a Binds to Human CD200R and to Cynomolgus CD200R

A BIACORE™ T200 (GE Healthcare, Piscataway, N.J.) is used to measure the binding kinetics and affinities of Antibody A to soluble human CD200R long isoform extracellular domain (ECD)-His polypeptide (SEQ ID NO: 17) (R&D Systems Cat. No. 10053-CD-050) and soluble cynomolgus monkey CD200R long isoform ECD-His polypeptide (SEQ ID NO: 19). Cynomolgus monkey CD200R ECD long isoform-His is expressed in HEK293 or CHO cells and purified using Ni SEPHAROSE™ excel column (GE Healthcare Life Sciences) and size exclusion chromatography.

A Series S CM4 chip (GE Healthcare Ca. No. BR-1005-34) is prepared using the manufacturer's EDC/NHS amine coupling method (GE Healthcare Cat. No. BR-1000-50). Briefly, the surfaces of all 4 flow cells are activated by injecting a 1:1 mixture of EDC/NHS for 7 minutes at 10 μL/minute. Protein A (Calbiochem Cat. No. 539202) is diluted to 100 μg/mL in 10 mM acetate pH 4.5 buffer and immobilized for approximately 400 RU onto all 4 flow cells by 7 minute injection at a flow rate of 10 μL/minute. Un-reacted sites are blocked with a 7 minute injection of ethanolamine at 10 μL/minute. Injections of 2×10 μL of glycine pH 1.5 are used to remove any non-covalently associated protein. Running buffer is 1× HBS-EP+ (10 mM HEPES, 150 mM NaCl, 0.05% Tween-20, pH 7.6, Teknova Cat. No. H8022).

Binding is evaluated using multi-cycle kinetics by an antibody capture method. Samples are diluted in 1× HBS-EP+ running buffer with 0.25% IgG free BSA (Jackson Immuno Research Cat. No. 001-000-161). Each cycle is performed at 37° C. at a flow rate of 20 μL/min for antibody capture to the Protein A chip and 30 μL/min for analyte association and dissociation. Each cycle consists of the following steps: injection of Antibody A at 2.5 μg/mL in HBS-EP+ with 0.25% IgG free BSA targeting antibody capture of 100 RU for human and 150 RU for cynomolgus on flow cell, injection of 700-seconds of analyte in HBS-EP+ with 0.25% IgG free BSA (concentration range of 2000 nM to 15.63 nM (human) or 1000 nM to 3.91 nM (cynomolgus) by two-fold serial dilution for human CD200R long isoform ECD-His and cynoCD200R long isoform ECD-His followed by 1800-second dissociation phase, and regeneration using two 25 μL injections of 10 mM glycine hydrochloride pH 1.5 over a 30-second contact time utilizing a 50 μL/min flow rate. All analyte concentrations are determined utilizing monomeric molecular weight (MW) values. Association rates (kₒₙ) and dissociation rates (k_{off}) for human CD200R long isoform ECD-His and cynomolgus CD200R long isoform ECD-His are evaluated using double referencing by flow-cell 1 reference subtraction in addition to 0 nM blank subtraction and fit to “1:1 (Langmuir) binding” model in the BIAevaluation software version 3.1. The dissociation constant (K_{D}) is calculated from the binding kinetics according to the relationship K_{D}=K_{off}/Kₒₙ. Stoichiometry=[RUₘₐₓ/RU_{captured}]/[MW_{CD200R ECD}/MW_{antibody}] where MW_{Antibody A} is 150 kDa and MW_{CD200R ECD} is approximately 28 kDa. Values are reported as mean±standard deviation.

In experiments performed essentially as described above, the results in Table 2 demonstrate that Antibody A binds to human CD200R-ECD long isoform-His (SEQ ID NO: 17) and cynoCD200R-ECD long isoform-His (SEQ ID NO: 19).

TABLE 2

| | On Rate (kₒₙ) | Off Rate (k_{off}) | Affinity (K_{D})^{a} | Stoichiometry |
|---|---|---|---|---|
| Species | (M⁻¹s⁻¹) (±SD) | (s⁻¹) (±SD) | (±SD) | (±SD) |
| Human | 4.7 ± 1.04 × 10³ | 1.6 ± 0.08 × 10⁻⁴ | 34.8 ± 10.4 nM | 1.9 ± 0.05 |
| CD200R Long | | | | |
| isoform ECD- | | | | |
| His (n = 3) | | | | |
| Cynomolgus | 6.2 ± 1.1 × 10³ | 5.6 ± 0.16 × 10⁻⁴ | 93.2 ± 15.3 nM | 2.6 ± 0.20 |
| CD200R Long | | | | |
| isoform ECD- | | | | |
| His (n = 3) | | | | |

^{a}Calculated as K_{D} = k_{off}/kₒₙ

### Antibody a Binds to Human CD200R Long Isoform, Human CD200R Short Isoform, Cynomolgus Monkey CD200R Long Isoform, and Cynomolgus Monkey CD200RLa in a Cell-Based Assay

CHO cells stably expressing human CD200R long isoform (SEQ ID NO: 15), human CD200R short isoform (SEQ ID NO: 16), cynomolgus monkey CD200R long isoform (SEQ ID NO: 18), and cynomolgus monkey CD200RLa (SEQ ID No: 20) are generated by transfection and selection (Rajendra Y, et al., Biotechnol. Prog 2017; 33, 534-40; Fan L, et al., J. Biotechnol. 2013; 168, 652-8; Fan L, et al., Biotechnol. Bioeng. 2012; 109: 1007-15).

To perform flow cytometry, CD200R-expressing CHO cells are counted using a Vicell counter and pelleted by centrifugation at 1200 RPM, aspirated and resuspended in phosphate buffered saline (PBS). Cells are counted again and after another centrifugation are adjusted to 10⁶ cells/mL in flow cytometry wash buffer (PBS w/10% NGS & 2% FBS & 0.05% NaN₃) with Fc block (BD Biosciences). Cells are blocked on ice for 15 minutes. Next the tubes are centrifuged, aspirated, and washed once with cold wash buffer. After the wash, the cells are adjusted to 10⁶ cells/mL in wash buffer and plated at 0.5⁶ cells/well (50 μL) in a 96-well tissue culture plate (Corning 3799). Antibody A is titrated (1:3 dilutions) on the 96-well polystyrene plates (Corning 3879) starting at 30 μg/mL (final concentration) in cold wash buffer. Next, Antibody A is transferred at 50 μL/well into the Corning 3799 plate containing cells. The plates are incubated on ice for 45 minutes then washed twice with 150 μL cold wash buffer. Next, 100 μL secondary Goat anti-human IgG Fc-PE (Jackson Cat. No. 109-116-098 or Jackson Cat. No. 109-116-088) is added at a 1:500 or 1:400 dilution in wash buffer and incubated for 45 minutes on ice. Plates are washed twice and 200 μL propidium iodide (1:1000 dilution in wash buffer; Molecular Probes Cat. No. P3566) is added as the cell viability stain. The plates are covered with foil on ice. Flow cytometry is then performed using a Fortessa instrument acquiring with FACS Diva software (both from Becton Dickinson) and analyzing with FlowJo (version 10.5.3).

Antibody A is tested for its binding to recombinantly expressed isoforms of both human and cynomolgus monkey CD200R and the related activating cynomolgus monkey CD200RLa protein on Chinese Hamster Ovary (CHO) cell line surfaces using flow cytometry. The antibody is titrated onto CHO cells expressing the polypeptide of interest. The flow cytometry binding curves are fit using the one-site saturation binding model in GraphPad Prism.

In experiments performed essentially as described above, and as shown in Table 3, Antibody A binds all four polypeptides, and exhibits similar titration midpoints (EC₅₀s) of binding to the different CD200R variants on CHO cells.

TABLE 3
Antibody A binding to polypeptides of interest

| Recombinant CD200R-expressing CHO | Midpoint of titration | |
|---|---|---|
| Cell Line | Antibody A (μg/mL) | R value |
| Human CD200R long isoform | 3.2 | 0.999 |
| Human CD200R short isoform | 1.1 | 0.999 |
| Cynomolgus monkey CD200R long | 2.3 | 0.996 |
| isoform | | |
| Cynomolgus monkey CD200RLa | 3.7 | 0.9999 |

### Antibody a Blocks Binding of Human CD200 to Human CD200R

The ability of Antibody A to block human CD200R and human CD200 ligand (“huCD200”) interaction can be measured using a whole-cell flow cytometry assay. A soluble huCD200-Fc chimeric protein (R&D Systems Cat. No. 2724-CD-050) is labeled with Alexa 647 dye (Thermo Fisher Scientific). When incubated with HEL92.1.7 cells expressing human CD200R at endogenous levels, the huCD200-Alexa 647 conjugate will bind the cell surface CD200R and that interaction results in Alexa 647 specific increases in fluorescence intensity which are measured by standard flow cytometry methods. Larger amounts of bound ligand lead to larger detectable fluorescent signals associated with measured cellular events. Likewise, blocking huCD200-Alexa 647 binding to CD200R decreases the detectable cell associated fluorescent signal.

HEL92.1.7 dead and live cells are differentially labeled with Biolegend ZOMBIE GREEN™ kit following manufacturer's standard protocol. ZOMBIE GREEN™ labeled HEL92.1.7 cells are then incubated on ice in PBS containing 1% BSA, 0.09% sodium azide and 200 ug/ml purified human IgG-PAA (“assay buffer”) for 30 minutes. Antibody A or control human IgG-PAA diluted in assay buffer are added to cells and then incubated on ice for an additional 90 minutes. At the end of the antibody incubation period the huCD200-Alexa 647 conjugate is diluted in the assay buffer, added to the sample and incubated on ice for a final 90 minutes. The concentration of huCD200-Alexa 647 in the final sample volume is approximately 1-2 ug/ml. The samples are then washed and fixed with paraformaldehyde prior to evaluation on a flow cytometer (BD Fortessa X-20 or similar). FCS files are analyzed with FlowJo software to delineate live cell events and their associated median fluorescence intensity in the Alexa 647 channel. The cell autofluorescence median intensity value for Alexa 647 channel is determined from samples without CD200-Alexa 647 addition and this value is considered background signal and subtracted from the evaluated sample values. Comparisons can be made between blocking antibodies and controls across an antibody concentration range. The CD200-Alexa 647 concentration is kept fixed across all samples (excluding background autofluorescence control samples). Median Fluorescence Intensity (MFI) values are exported from FlowJo and technical replicate mean and standard deviation values are determined using GraphPad Prism or MS Excel Software tools.

In experiments performed essentially as described above, Antibody A blocks huCD200-Alexa 647 binding to HEL92.1.7 cells in a concentration dependent manner (Table 4).

TABLE 4

| | | | |
|---|---|---|---|
| Concentration of | 100 | 33.3 | 11.1 |
| Antibody (nM) | | | |
| IgG4-PAA | 465.5 | 470.5 | 471.5 |
| Control (MFI) | | | |
| Antibody A | 46.5 | 181.0 | 341.0 |
| (MFI) | | | |

### Antibody a Antagonizes Human CD200R in a Cell Based Assay

The PATHHUNTER® dimerization assay (DiscoverX, Fremont, Calif.) detects the interaction between huCD200 and its receptor CD200R. Ligation of the receptor leads to assembly of B-galactosidase and a subsequent luminescent readout. Blocking that interaction with an anti-CD200R antibody will decrease or abrogate the luminescent signal. CD200R-expressing Jurkat cells are in incubated with titrating concentrations of Antibody A for 1 hour at 37° C. in a 384-well plate format. U2OS-CD200 cells are added at a 3:1 ratio to Jurkat-CD200R cells and incubated for 2 hour at room temperature. After incubation, PATHHUNTER® Flash Detection Kit reagent is added to the plate(s) and incubated for an additional 30 min at room temperature protected from light. Luminescence is measured on the BioTek Synergy Neo 2 plate reader (BioTek Instruments, Winoosky, Vt.).

Data are normalized using 2-point normalization (1-(Value-Median of Min)/(Median of Max-Median of Min)×100%; Min=column 1 from plate (no CD200-expressing cells), Max=column 24 from plate (CD200-expressing cells, no treatment) and reported as a % activity. The results indicate that Antibody A blocks the interaction between huCD200 and human CD200R in this reporter assay system.

Upon CD200 ligand binding, CD200R is phosphorylated on the tyrosine of the NPXY motif and subsequently binds adapter proteins Dok1 and Dok2. Phosphorylation of these adapter proteins recruits SHIP and RasGAP, which subsequently inhibits the Ras/MAPK activation pathways (Zhang, S. et al., J. Immunol. 2004; 173: 6786-6793). Blocking CD200 ligand signaling with Antibody A can be evaluated by measuring the level of Dok2 phosphorylation relative to the level of Dok2 phosphorylation observed in a control experiment performed in the absence of the CD200R antagonist. The level of Dok2 phosphorylation is expected to be reduced if huCD200 binding is blocked/inhibited by treatment with Antibody A.

Human primary macrophages are generated by culturing fresh human PBMC (obtained from a healthy donor from New York Blood Bank or Leukopak) in complete IMDM medium in the presence of 40 ng/mL human M-CSF and 20 ng/ml hIL-4 for 8 days in dishes coated with 10 ug/mL fibronectin. U2OS-parental or human-CD200-transformed U2OS cells are grown in 0.25 ug/mL puromycin in McCoy's 5a+10% fetal bovine serum+1× glutamax. U2OS-parental or U2OS-CD200 cells are harvested and added to the IL-4 matured macrophages in a 1:1 ratio for 20 minutes (serum-free RPMI+glutamax) with the IgG4-PAA control. Cells culture supernatants are removed and cells are lysed and prepared for Western blotting.

In experiments performed essentially as described above, the addition of Antibody A nearly completely inhibits the phosphorylation of Dok2 in IL-4 induced human primary macropahges stimulated with U2OS-CD200L cells, as compared to the level of phosphorylation observed for the IgG4-PAA control. The results indicate that Antibody A is a CD200R antagonist molecule.

### Antibody A Does Not Elicit Mast Cell Degranulation In Vitro

Cynomolgus monkey CD200RLa (cynoCD200RLa) is an activating receptor and is expressed in mast cells (Zhang S and J H Phillips, J. Leukocyte Biol. 2005; 79: 363-368). A mast cell degranulation assay tests whether a polypeptide molecule will bind to cynoCD200RLa and potentiate mast cell activation/degranulation. Briefly, MC/9 mouse mast cells transduced to express cynoCD200RLa are stimulated with Antibody A, with or without a cross-linking Fab (F(ab′)2-goat anti-human IgG Fc-gamma secondary antibody (Invitrogen Cat. No. 31163)), for 18-24 hours. Culture supernatants are analyzed via ELISA for mIL-13 and mTNFα release, which indicate mast cell activation/degranulation (Theoharides T C, et al., Biochim. Biophys. Acta 2013; 1822: 21-33).

As shown in Table 5, in the absence of the cross-linking Fab, the amount of each of mIL-13 and mTNFα secreted from MC/9-cynoCD200RLa expressing cells after treatment with Antibody A is substantially the same, respectively, as the amount of mIL-13 and mTNFα secreted from MC/9-cynoCD200RLa expressing cells after treatment with the cross-linking Fab alone, and is a fraction of the amount each of mIL-13 and mTNFα, respectively, that is secreted from each of (a) MC/9-cynoCD200RLa expressing cells after treatment with calcium ionophore A23187 (10 mg, MW: 528.13 g/mol, Sigma), which is known to elicit mast cell activation/degranulation (Pearce F L, Br. J. Clin. Pharm. 1985; 20: 267S-274S)), and (b) MC/9-cynoCD200RLa expressing cells after treatment with Antibody A and the cross-linking Fab.

TABLE 5

| | MC/9 Parental | | MC/9-cynoCD200RLa | |
|---|---|---|---|---|
| | mIL-13 | mTNFα | mIL-13 | mTNFa |
| | (pg/mL) | (pg/mL) | (pg/mL) | (pg/mL) |
| Antibody A | 9.7 | 0 | 133.3 | 15.4 |
| Antibody A + Cross-Linking | 68.2 | 0 | 4698 | 2490.5 |
| Fab | | | | |
| Cross-Linking Fab alone | 10.8 | 0 | 117.1 | 15.3 |
| Calcium Ionophore A23187 | 151 | 2692.5 | 4698 | 1173 |

Antibody a Demonstrates Antitumor Efficacy in the HCC827 NSCLC Tumor Xenograft Model Infused with Human T Cells in NSG Mice.

On Day 0, 10×10⁶ HCC827 cells are resuspended in 0.2 mL HBSS and subcutaneously implanted into the right flank of thirty-two (32) female NOD/SCID Gamma (NSG) mice (Jackson Laboratories). On Day 35, mice are randomized at n=8 and dosed intraperitoneally (ip) at 10 mg/kg once a week for 3 weeks per treatment group. On the day of randomization, twenty-four (24) mice were infused with 3M T cells per mouse concentration. Treatment groups include a control IgG-effector null (EN) (which has the same framework as Antibody A), an anti-PD-L1-IgG-EN antibody (Li Y, et al., J. ImmunoTherapy of Cancer 2018; 6: 31-44), and Antibody A.

Body weight and tumor volume are measured twice a week. Tumor volume (mm³) is calculated as π/6*Length*Width² and % T/C is calculated as 100×ΔT/ΔC, if ΔT>0 of the geometric mean values. Statistical analysis is performed using the procedures in the SAS software.

In experiments performed essentially as described above on day 51, the results in Table 6 demonstrate that the positive control anti-PD-L1-IgG-EN antibody dosed at 10 mg/kg significantly inhibits tumor growth by 26.4% (P<0.01), and Antibody A dosed at 10 mg/kg results in a % tumor growth inhibition of approximately 42.8% (p<0.006) in the human T Cell infused mice, relative to the control IgG-EN treated group.

TABLE 6

| | | p-value for | |
|---|---|---|---|
| | Xenograft | tumor volume | % T/C |
| Control IgG-EN 10 mg/kg | HCC827 | 0.226 | 134.9% |
| | unengrafted | | |
| Control IgG-EN 10 mg/kg | HCC827 | NA | NA |
| Anti-PD-L1-IgG-EN 10 mg/kg | HCC827 | <0.001 | 26.4% |
| Antibody A 10 mg/kg | HCC827 | 0.006 | 42.8% |

Amino Acid and Nucleotide Sequences

SEQ ID NO: 1 (HCDR1 amino acid sequence)
AASGFTFSRYGMH
SEQ ID NO: 2 (HCDR2 amino acid sequence)
VIPYDGSNKY
SEQ ID NO: 3 (HCDR3 amino acid sequence)
ARRGYYDSSGYYYFYYGMDV
SEQ ID NO: 4 (LCDR1 amino acid sequence)
RASQSVSSNLA
SEQ ID NO: 5 (LCDR2 amino acid sequence)
YGASTRAT
SEQ ID NO: 6 (LCDR3 amino acid sequence)
QQYNKWPPIT
SEQ ID NO: 7 (HCVR amino acid sequence)
QVQLVESGGGVVQPGRSLRLSCAASGFTFSRYGMHWVRQAPGKGLEWVAVIPY
DGSNKYYADSVKGRFTISRDISKNTLYLQMNSLRAEDTAVYYCARRGYYDSSGY
YYFYYGMDVWGQGTTVTVSS
SEQ ID NO: 8 (LCVR amino acid sequence)
EIVMTQSPATLSVSPGERATLSCRASQSVSSNLAWYQQKPGQAPRLLIYGASTRA
TGIPARFSGSGSGTEFTLTISSLQSEDFAVYYCQQYNKWPPITFGGGTKVEIK
SEQ ID NO: 9 (HCCR amino acid sequence)
ASTKGPSVFPLAPCSRSTSESTAALGCLVKDYFPEPVTVSWNSGALTSGVHTFPA
VLQSSGLYSLSSVVTVPSSSLGTKTYTCNVDHKPSNTKVDKRVESKYGPPCPPCP
APEAAGGPSVFLFPPKPKDTLMISRTPEVTCVVVDVSQEDPEVQFNWYVDGVEV
HNAKTKPREEQFNSTYRVVSVLTVLHQDWLNGKEYKCKVSNKGLPSSIEKTISK
AKGQPREPQVYTLPPSQEEMTKNQVSLTCLVKGFYPSDIAVEWESNGQPENNYK
TTPPVLDSDGSFFLYSRLTVDKSRWQEGNVFSCSVMHEALHNHYTQKSLSLSLG
SEQ ID NO: 10 (LCCR amino acid sequence)
RTVAAPSVFIFPPSDEQLKSGTASVVCLLNNFYPREAKVQWKVDNALQSGNSQES
VTEQDSKDSTYSLSSTLTLSKADYEKHKVYACEVTHQGLSSPVTKSFNRGEC
SEQ ID NO: 11 (Heavy Chain amino acid sequence)
QVQLVESGGGVVQPGRSLRLSCAASGFTFSRYGMHWVRQAPGKGLEWVAVIPY
DGSNKYYADSVKGRFTISRDISKNTLYLQMNSLRAEDTAVYYCARRGYYDSSGY
YYFYYGMDVWGQGTTVTVSSASTKGPSVFPLAPCSRSTSESTAALGCLVKDYFP
EPVTVSWNSGALTSGVHTFPAVLQSSGLYSLSSWTVPSSSLGTKTYTCNVDHKP
SNTKVDKRVESKYGPPCPPCPAPEAAGGPSVFLFPPKPKDTLMISRTPEVTCVVVD
VSQEDPEVQFNWYVDGVEVHNAKTKPREEQFNSTYRVVSVLTVLHQDWLNGKE
YKCKVSNKGLPSSIEKTISKAKGQPREPQVYTLPPSQEEMTKNQVSLTCLVKGFY
PSDIAVEWESNGQPENNYKTTPPVLDSDGSFFLYSRLTVDKSRWQEGNVFSCSV
MHEALHNHYTQKSLSLSLG
SEQ ID NO: 12 (Light Chain amino acid sequence)
EIVMTQSPATLSVSPGERATLSCRASQSVSSNLAWYQQKPGQAPRLLIYGASTRA
TGIPARFSGSGSGTEFTLTISSLQSEDFAVYYCQQYNKWPPITFGGGTKVEIKRTV
AAPSVFIFPPSDEQLKSGTASVVCLLNNFYPREAKVQWKVDNALQSGNSQESVTE
QDSKDSTYSLSSTLTLSKADYEKHKVYACEVTHQGLSSPVTKSFNRGEC
SEQ ID NO: 13 (Heavy Chain DNA sequence)
CAGGTGCAGCTGGTGGAGTCTGGGGGAGGCGTGGTCCAGCCTGGGAGGTCCC
TGAGACTCTCCTGTGCAGCCTCTGGATTCACTTTCAGTAGATATGGCATGCAC
TGGGTCCGCCAGGCTCCAGGCAAGGGGCTGGAGTGGGTGGCAGTTATACCAT
ATGATGGAAGTAATAAATACTATGCAGACTCCGTGAAGGGCCGATTCACCAT
CTCCAGAGACATTTCCAAGAACACACTGTATCTGCAAATGAACAGCCTGAGA
GCTGAGGACACGGCTGTGTATTACTGTGCGAGAAGGGGGTACTATGATAGTA
GTGGTTATTACTACTTCTACTACGGTATGGACGTCTGGGGCCAAGGGACCACG
GTCACCGTCTCCTCAGCTTCTACCAAGGGCCCATCGGTCTTCCCGCTAGCGCC
CTGCTCCAGGAGCACCTCCGAGAGCACAGCCGCCCTGGGCTGCCTGGTCAAG
GACTACTTCCCCGAACCGGTGACGGTGTCGTGGAACTCAGGCGCCCTGACCA
GCGGCGTGCACACCTTCCCGGCTGTCCTACAGTCCTCAGGACTCTACTCCCTC
AGCAGCGTGGTGACCGTGCCCTCCAGCAGCTTGGGCACGAAGACCTACACCT
GCAACGTAGATCACAAGCCCAGCAACACCAAGGTGGACAAGAGAGTTGAGT
CCAAATATGGTCCCCCATGCCCACCCTGCCCAGCACCTGAGGCCGCCGGGGG
ACCATCAGTCTTCCTGTTCCCCCCAAAACCCAAGGACACTCTCATGATCTCCC
GGACCCCTGAGGTCACGTGCGTGGTGGTGGACGTGAGCCAGGAAGACCCCGA
GGTCCAGTTCAACTGGTACGTGGATGGCGTGGAGGTGCATAATGCCAAGACA
AAGCCGCGGGAGGAGCAGTTCAACAGCACGTACCGTGTGGTCAGCGTCCTCA
CCGTCCTGCACCAGGACTGGCTGAACGGCAAGGAGTACAAGTGCAAGGTCTC
CAACAAAGGCCTCCCGTCCTCCATCGAGAAAACCATCTCCAAAGCCAAAGGG
CAGCCCCGAGAGCCACAGGTGTACACCCTGCCCCCATCCCAGGAGGAGATGA
CCAAGAACCAGGTCAGCCTGACCTGCCTGGTCAAAGGCTTCTACCCCAGCGA
CATCGCCGTGGAGTGGGAAAGCAATGGGCAGCCGGAGAACAACTACAAGAC
CACGCCTCCCGTGCTGGACTCCGACGGCTCCTTCTTCCTCTACAGCAGGCTAA
CCGTGGACAAGAGCAGGTGGCAGGAGGGGAATGTCTTCTCATGCTCCGTGAT
GCATGAGGCTCTGCACAACCACTACACACAGAAGAGCCTCTCCCTGTCTCTGG
GT
SEQ ID NO: 14 (Light Chain DNA sequence)
GAAATTGTGATGACGCAGTCTCCAGCCACCCTGTCTGTGTCTCCA
GGGGAAAGAGCCACCCTCTCCTGCAGGGCCAGTCAGAGTGTTAG
CAGCAACTTAGCCTGGTACCAGCAGAAACCTGGCCAGGCTCCCAG
GCTCCTCATCTATGGTGCATCCACCAGGGCCACTGGTATCCCAGC
CAGGTTCAGTGGCAGTGGGTCTGGGACAGAGTTCACTCTCACCAT
CAGCAGCCTGCAGTCTGAAGATTTTGCAGTTTATTACTGTCAGCA
GTATAATAAGTGGCCTCCGATCACTTTCGGCGGAGGGACCAAGGT
GGAGATCAAACGGACCGTGGCTGCACCATCTGTCTTCATCTTCCC
GCCATCTGATGAGCAGTTGAAATCTGGAACTGCCTCTGTTGTGTG
CCTGCTGAATAACTTCTATCCCAGAGAGGCCAAAGTACAGTGGAA
GGTGGATAACGCCCTCCAATCGGGTAACTCCCAGGAGAGTGTCAC
AGAGCAGGACAGCAAGGACAGCACCTACAGCCTCAGCAGCACCC
TGACGCTGAGCAAAGCAGACTACGAGAAACACAAAGTCTACGCC
TGCGAAGTCACCCATCAGGGCCTGAGCTCGCCCGTCACAAAGAGC
TTCAACAGGGGAGAGTGC
SEQ ID NO: 15 (Human CD200R long isoform amino acid sequence)
MLCPWRTANLGLLLILTIFLVAEAEGAAQPNNSLMLQTSKENHALASSSLCMDE
KQITQNYSKVLAEVNTSWPVKMATNAVLCCPPIALRNLIIITWEIILRGQPSCTKA
YRKETNETKETNCTDERITWVSRPDQNSDLQIRPVAITHDGYYRCIMVTPDGNFH
RGYHLQVLVTPEVTLFQNRNRTAVCKAVAGKPAAQISWIPEGDCATKQEYWSN
GTVTVKSTCHWEVHNVSTVTCHVSHLTGNKSLYIELLPVPGAKKSAKLYIPYIILT
IIILTIVGFIWLLKVNGCRKYKLNKTESTPVVEEDEMQPYASYTEKNNPLYDTTNK
VKASQALQSEVDTDLHTL
SEQ ID NO: 16 (Human CD200R short isoform amino acid sequence)
METDTLLLWVLLLWVPGSTGASSSLCMDEKQITQNYSKVLAEVNTSWPVKMAT
KAVLCCPPIALRNLIIITWEIILRGQPSCTKAYKKETNETKETNCTDERITWVSRPD
QNSDLQIRTVAITHDGYYRCIMVTPDGNFHRGYHLQVLVTPEVTLFQNRNRTAV
CKAVAGKPAAHISWIPEGDCATKQEYWSNGTVTVKSTCHWEVHNVSTVTCHVS
HLTGNKSLYIELLPVPGAKKSAKLYIPYIILTIIILTIVGFIWLLKVNGCRKYKLNKT
ESTPVVEEDEMQPYASYTEKNNPLYDTTNKVKASEALQSEVDTDLHTL
SEQ ID NO: 17 (Human CD200R long isoform ECD-His amino acid sequence)
AAQPNNSLMLQTSKENHALASSSLCMDEKQITQNYSKVLAEVNTSWPVKMATN
AVLCCPPIALRNLIIITWEIILRGQPSCTKAYKKETNETKETNCTDERITWVSRPDQ
NSDLQIRTVAITHDGYYRCIMVTPDGNFHRGYHLQVLVTPEVTLFQNRNRTAVC
KAVAGKPAAHISWIPEGDCATKQEYWSNGTVTVKSTCHWEVHNVSTVTCHVSH
LTGNKSLYIELLPVPGAKKSAKLHHHHHH
SEQ ID NO: 18 (Cynomolgus monkey CD200R long isoform amino acid sequence)
MLCPWRTANLGLLLILAVFLVAEAEGAAQSNNSLMLQTSKENHTLASNSLCMDE
KQITQNHSKVLAEVNISWPVQMARNAVLCCPPIEFRNLIVITWEIILRGQPSCTKSY
RKETNETKEINCTDERITWVSTPDQNSDLQIHPVAITHDGYYRCIMATPDGNFHR
GYHLQVLVTPEVTLFESRNRTAVCKAVAGKPAAQISWIPAGDCAPTEQEYWGNG
TVTVKSTCHWEGHNVSTVTCHVSHLTGNKSLYIELLPVPGAKKSAKLYMPYVIL
TIIILTIVGFIWLLKISGCRKYNLNKTESTSVVEEDEMQPYASYTEKNNPLYDTTNK
VKASQALQSEVGTDLHTL
SEQ ID NO: 19 (Cynomolgus Monkey CD200R long isoform ECD-His amino acid
sequence)
EGAAQSNNSLMLQTSKENHTLASNSLCMDEKQITQNHSKVLAEVNISWPVQMA
RNAVLCCPPIEFRNLIVITWEIILRGQPSCTKTYRKDTNETKETNCTDERITWVSTP
DQNSDLQIHPVAITHDGYYRCIMATPDGNFHRGYHLQVLVTPEVTLFESRNRTAV
CKAVAGKPAAQISWIPAGDCAPTEQEYWGNGTVTVKSTCHWEGHNVSTVTCHV
SHLTGNKSLYIELLPVPGAKKSAKGSHHHHHHHH
SEQ ID NO: 20 (Cynomolgus Monkey CD200RLa amino acid sequence)
METDTLLLWVLLLWVPGSTGSSCMDGKQMTQNYSKMSAEGNISQPVLMDTNA
MLCCPPIEFRNLIVIVWEIIIRGQPSCTKAYRKETNETKETNCTDERITWVSTPDQN
SDLQIHPVAITHDGYYRCIMATPDGNFHRGYHLQVLVTPEVTLFQSRNRTAVCKA
VAGKPAAQISWIPAGDCAPTEHEYWGNGTVTVESMCHWGDHNASTMTCHVSH
LTGNKSLYIKLNSGLRTSGSPALDLLIILYVKLSLFVVILVTTGFVFFQRINYVRKS
L

1. A polypeptide molecule that binds to the human CD200R long and short isoforms, wherein the polypeptide molecule comprises each of the amino acid sequences of SEQ ID NOS: 1-6.

2. The polypeptide molecule of claim 1, wherein the polypeptide molecule is an scFv.

3. The polypeptide molecule of claim 1, wherein the polypeptide molecule is a Fab.

4. The polypeptide molecule of claim 2, wherein the polypeptide molecule is an antibody comprising:
a) a heavy chain comprising an HCDR1 having the amino acid sequence of SEQ ID NO: 1, an HCDR2 having the amino acid sequence of SEQ ID NO: 2, and an HCDR3 having the amino acid sequence of SEQ ID NO: 3; and
b) a light chain comprising an LCDR1 having the amino acid sequence of SEQ ID NO: 4, an LCDR2 having the amino acid sequence of SEQ ID NO: 5, and an LCDR3 having the amino acid sequence of SEQ ID NO: 6.

5. The polypeptide molecule of claim 4, wherein the antibody is a mono-specific antibody.

6. The polypeptide molecule of claim 4, wherein the antibody is a polyspecific antibody.

7. The polypeptide molecule of claim 6, wherein the antibody is a bispecific antibody.

8. The polypeptide molecule of claim 4, wherein the antibody comprises a heavy chain variable region having the amino acid sequence of SEQ ID NO: 7 and a light chain variable region having the amino acid sequence of SEQ ID NO: 8.

9. The polypeptide molecule of claim 8, wherein the polypeptide molecule is an antibody comprising a heavy chain having the amino acid sequence of SEQ ID NO: 11 and a light chain having the amino acid sequence of SEQ ID NO: 12.

10. A mammalian cell capable of expressing the polypeptide molecule of claim 4.

11. A polynucleotide molecule comprising a polynucleotide encoding one or both of the polypeptide molecule amino acid sequences of SEQ ID NO: 11 and SEQ ID NO: 12.

12. The polynucleotide molecule of claim 11, wherein the polynucleotide comprises one or both of the polynucleotides of SEQ ID NO: 13 and SEQ ID NO: 14.

13. (canceled)

14. A process for producing a polypeptide molecule, comprising cultivating the mammalian cell of claim 10, and recovering the polypeptide molecule.

15. The polypeptide molecule produced by the method of claim 14.

16. A pharmaceutical composition comprising the polypeptide molecule of claim 4, and an acceptable carrier, diluent, or excipient.

17. A method of treating a solid tumor cancer, liquid tumor cancer or neuroendocrine tumor cancer comprising administering to a human patient in need thereof, an effective amount of the polypeptide molecule of claim 1.

18. The method of claim 17, wherein the solid tumor cancer is breast cancer, bladder cancer, cervical cancer, colorectal cancer, endometrial cancer, gastric cancer, head and neck cancer, hepatocellular carcinoma, liver cancer, lung cancer, melanoma, pancreatic cancer, prostate cancer, ovarian cancer, renal cancer, testicular cancer, or thyroid cancer.

19. The method of claim 18, wherein the solid tumor cancer is lung cancer, breast cancer or pancreatic cancer.

20. The method of claim 17, wherein the liquid tumor cancer is B-cell lymphoma, T-cell lymphoma, leukemia, Hodgkin lymphoma, myeloma, myelodysplasic syndrome, or plasmacytoma.

21. The method of claim 20, wherein the leukemia is chronic lymphocytic leukemia or acute myeloid leukemia.

22. The method of claim 17, wherein the neuroendocrine tumor cancer is large cell neuroendocrine cancer or pancreatic neuroendocrine cancer.

23. The method of any one of claim 17, wherein the polypeptide molecule is administered in simultaneous, separate, or sequential combination with one or more immuno-oncology agents.

24. The method of claim 23, wherein the immuno-oncology agent is an anti-PD-1 antibody or an anti-PD-L1 antibody.

25.-41. (canceled)


SEQUENCE LISTING (20 sequences)

SEQ ID NO: 1 - protein, 13 aa, Artificial Sequence
  Synthetic Construct
AASGFTFSRYGMH

SEQ ID NO: 2 - protein, 10 aa, ARTIFICIAL SEQUENCE
  Synthetic Construct
VIPYDGSNKY

SEQ ID NO: 3 - protein, 20 aa, ARTIFICIAL SEQUENCE
  Synthetic Construct
ARRGYYDSSGYYYFYYGMDV

SEQ ID NO: 4 - protein, 11 aa, ARTIFICIAL SEQUENCE
  Synthetic Construct
RASQSVSSNLA

SEQ ID NO: 5 - protein, 8 aa, ARTIFICIAL SEQUENCE
  Synthetic Construct
YGASTRAT

SEQ ID NO: 6 - protein, 10 aa, ARTIFICIAL SEQUENCE
  Synthetic Construct
QQYNKWPPIT

SEQ ID NO: 7 - protein, 127 aa, ARTIFICIAL SEQUENCE
  Synthetic Construct
QVQLVESGGGVVQPGRSLRLSCAASGFTFSRYGMHWVRQAPGKGLEWVAVIPYDGSNKYYADSVKGRFTISRDISKNTLYLQMNSLRAEDTAVYYCARRGYYDSSGYYYFYYGMDVWGQGTTVTVSS

SEQ ID NO: 8 - protein, 108 aa, ARTIFICIAL SEQUENCE
  Synthetic Construct
EIVMTQSPATLSVSPGERATLSCRASQSVSSNLAWYQQKPGQAPRLLIYGASTRATGIPARFSGSGSGTEFTLTISSLQSEDFAVYYCQQYNKWPPITFGGGTKVEIK

SEQ ID NO: 9 - protein, 326 aa, ARTIFICIAL SEQUENCE
  Synthetic Construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 10 - protein, 107 aa, ARTIFICIAL SEQUENCE [sequence omitted]

SEQ ID NO: 11 - protein, 453 aa, ARTIFICIAL SEQUENCE
  Synthetic Construct
QVQLVESGGGVVQPGRSLRLSCAASGFTFSRYGMHWVRQAPGKGLEWVAVIPYDGSNKYYADSVKGRFTISRDISKNTLYLQMNSLRAEDTAVYYCARRGYYDSSGYYYFYYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 12 - protein, 215 aa, ARTIFICIAL SEQUENCE
  Synthetic Construct
EIVMTQSPATLSVSPGERATLSCRASQSVSSNLAWYQQKPGQAPRLLIYGASTRATGIPARFSGSGSGTEFTLTISSLQSEDFAVYYCQQYNKWPPITFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - DNA, 1359 bp, ARTIFICIAL SEQUENCE
  Synthetic Construct
caggtgcagctggtggagtctgggggaggcgtggtccagcctgggaggtccctgagactctcctgtgcagcctctggattcactttcagtagatatggcatgcactgggtccgccaggctccaggcaaggggctggagtgggtggcagttataccatatgatggaagtaataaatactatgcagactccgtgaagggccgattcaccatctccagagacatttccaagaacacactgtatctgcaaatgaacagcctgagagctgaggacacggctgtgtattactgtgcgagaagggggtactatgatagtagtggttattactacttctactacggtatggacgtctggggccaagggaccacggtcaccgtctcctcagcttctaccaagggcccatcggtcttcccgctagcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgaggccgccgggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggaaagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggt

SEQ ID NO: 14 - DNA, 645 bp, ARTIFICIAL SEQUENCE
  Synthetic Construct
gaaattgtgatgacgcagtctccagccaccctgtctgtgtctccaggggaaagagccaccctctcctgcagggccagtcagagtgttagcagcaacttagcctggtaccagcagaaacctggccaggctcccaggctcctcatctatggtgcatccaccagggccactggtatcccagccaggttcagtggcagtgggtctgggacagagttcactctcaccatcagcagcctgcagtctgaagattttgcagtttattactgtcagcagtataataagtggcctccgatcactttcggcggagggaccaaggtggagatcaaacggaccgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgc

SEQ ID NO: 15 - protein, 348 aa, homo sapiens [sequence omitted]

SEQ ID NO: 16 - protein, 323 aa, homo sapiens
METDTLLLWVLLLWVPGSTGASSSLCMDEKQITQNYSKVLAEVNTSWPVKMATKAVLCCPPIALRNLIIITWEIILRGQPSCTKAYKKETNETKETNCTDERITWVSRPDQNSDLQIRTVAITHDGYYRCIMVTPDGNFHRGYHLQVLVTPEVTLFQNRNRTAVCKAVAGKPAAHISWIPEGDCATKQEYWSNGTVTVKSTCHWEVHNVSTVTCHVSHLTGNKSLYIELLPVPGAKKSAKLYIPYIILTIIILTIVGFIWLLKVNGCRKYKLNKTESTPVVEEDEMQPYASYTEKNNPLYDTTNKVKASEALQSEVDTDLHTL

SEQ ID NO: 17 - protein, 246 aa, ARTIFICIAL SEQUENCE
  Synthetic Construct
AAQPNNSLMLQTSKENHALASSSLCMDEKQITQNYSKVLAEVNTSWPVKMATNAVLCCPPIALRNLIIITWEIILRGQPSCTKAYKKETNETKETNCTDERITWVSRPDQNSDLQIRTVAITHDGYYRCIMVTPDGNFHRGYHLQVLVTPEVTLFQNRNRTAVCKAVAGKPAAHISWIPEGDCATKQEYWSNGTVTVKSTCHWEVHNVSTVTCHVSHLTGNKSLYIELLPVPGAKKSAKLHHHHHH

SEQ ID NO: 18 - protein, 349 aa, Macaca fascicularis
MLCPWRTANLGLLLILAVFLVAEAEGAAQSNNSLMLQTSKENHTLASNSLCMDEKQITQNHSKVLAEVNISWPVQMARNAVLCCPPIEFRNLIVITWEIILRGQPSCTKSYRKETNETKEINCTDERITWVSTPDQNSDLQIHPVAITHDGYYRCIMATPDGNFHRGYHLQVLVTPEVTLFESRNRTAVCKAVAGKPAAQISWIPAGDCAPTEQEYWGNGTVTVKSTCHWEGHNVSTVTCHVSHLTGNKSLYIELLPVPGAKKSAKLYMPYVILTIIILTIVGFIWLLKISGCRKYNLNKTESTSVVEEDEMQPYASYTEKNNPLYDTTNKVKASQALQSEVGTDLHTL

SEQ ID NO: 19 - protein, 252 aa, ARTIFICIAL SEQUENCE
  Synthetic Construct
EGAAQSNNSLMLQTSKENHTLASNSLCMDEKQITQNHSKVLAEVNISWPVQMARNAVLCCPPIEFRNLIVITWEIILRGQPSCTKTYRKDTNETKETNCTDERITWVSTPDQNSDLQIHPVAITHDGYYRCIMATPDGNFHRGYHLQVLVTPEVTLFESRNRTAVCKAVAGKPAAQISWIPAGDCAPTEQEYWGNGTVTVKSTCHWEGHNVSTVTCHVSHLTGNKSLYIELLPVPGAKKSAKGSHHHHHHHH

SEQ ID NO: 20 - protein, 274 aa, Macaca fascicularis
METDTLLLWVLLLWVPGSTGSSCMDGKQMTQNYSKMSAEGNISQPVLMDTNAMLCCPPIEFRNLIVIVWEIIIRGQPSCTKAYRKETNETKETNCTDERITWVSTPDQNSDLQIHPVAITHDGYYRCIMATPDGNFHRGYHLQVLVTPEVTLFQSRNRTAVCKAVAGKPAAQISWIPAGDCAPTEHEYWGNGTVTVESMCHWGDHNASTMTCHVSHLTGNKSLYIKLNSGLRTSGSPALDLLIILYVKLSLFVVILVTTGFVFFQRINYVRKSL